(19) United States
(12) Patent Application Publication
(10) Pub. No.: US 2002/0182663 A1
(43) Pub. Date: Dec. 5, 2002

(54) Identification and enumeration of coccidial sporocysts

(75) Inventor: Mary Ann Pfannenstiel, Lincoln, NE (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Novus International, Inc.

(21) Appl. No.: 10/107,932

(22) Filed: Mar. 27, 2002

Related U.S. Application Data
(60) Provisional application No. 60/278,974, filed on Mar. 27, 2001.

(51) Int. Cl. G01N 33/53; G01N 33/569; G06F 19/00

(52) U.S. Cl. 435/34; 702/21

(57) ABSTRACT

A method for the identification and enumeration by flow cytometry of species of coccidial sporocysts and in particular sporocysts of the genus Eimeria is disclosed. The method is useful in the identification and enumeration of coccidial protozoa in environmental samples, designing treatment programs for animals infected with coccidial protozoa, and for quality control of preparations containing coccidial protozoa.

### CROSS-REFERENCE TO RELATED APPLICATIONS

This application claims priority from U.S. Provisional Application Serial No. 60/278,974, filed Mar. 27, 2001, which is herein incorporated by reference in its entirety for all purposes.

### BACKGROUND

Coccidiosis is a disease of various animals in which the intestinal mucosa is invaded and damaged by protozoa of the subclass Coccidia. The economic effects of coccidiosis can be especially severe in the poultry industry where intensive housing of birds favors the spread of the disease. Infection by coccidial protozoa is, for the most part, species specific. Numerous species, however, can infect a single host. For example, there are seven species of Coccidia which infect chickens, six of which are considered to be moderately to severely pathogenic.

The life cycle of the coccidial parasite is complex. For example, protozoa of the genera Eimeria, Isospora, Cystoisospora, or Cryptosporidium typically only require a single host to complete their life cycle, although Cystoisospora may utilize an intermediate host. Under natural conditions, the life cycle begins with the ingestion of sporulated oocysts from the environment. When sporulated oocysts are ingested by a susceptible animal, the wall of the sporulated oocyst is broken in order to release the sporocysts inside. In poultry, the release of the sporocyst is the result of mechanical disruption of the sporulated oocyst in the gizzard. Within the sporocysts are the sporozoites which are the infective stage of the organism. In poultry, the breakdown of the sporocyst coat and release of the sporozoites is accomplished biochemically through the action of chymotrypsin and bile salts in the small intestine. Once released, the sporozoites invade the intestinal mucosa or epithelial cells in other locations. The site of infection is characteristic of the species involved. For example, in the genus Eimeria, E. tenella is localized in the ceca; E. necatrix is found in the anterior and middle portions of the small intestine; E. acervulina and E. praecox occur in the upper half of the small intestine; E. brunetti occurs in the lower small intestine, rectum, ceca, and cloaca; while E. mitis are found in the lower small intestine.

Once inside the host animal's cells, sporozoites develop into multinucleate meronts, also called schizonts. Each nucleus of the meront develops into an infective body called a merozoite which enters new cells and repeats the process. After a variable number of asexual generations, merozoites develop into either microgametocytes or macrogametes. Microgametocytes develop into many microgametes which, in turn, fertilize the macrogametes. A resistant coat then forms around the resulting zygotes. The encysted zygotes, called oocysts, are shed unsporulated in the feces. Infected birds may shed oocysts in the feces for days or weeks. Under proper conditions of temperature and moisture, the oocysts become infective through the process of sporulation. Susceptible birds then ingest the sporulated oocysts through normal pecking activities and the cycle repeats itself. Ingestion of viable, sporulated oocysts is the only natural means of infection.

Infection with coccidial protozoa results in immunity so that the incidence of the disease decreases over time as members of the flock become immune. This self-limiting nature of coccidial infections is widely known in chickens and other poultry. The immunity conferred, however, is species specific such that introduction of another species of coccidial protozoa will result in a new disease outbreak.

Two methods are currently used to control coccidiosis in poultry. The first involves control by chemotherapy. Numerous drugs are available for the control of coccidiosis in poultry. Because of the number of species which cause the disease, very few drugs are efficacious against all species, although a single drug may be efficacious against several species. In modern broiler chicken production, for example, administration of drugs to control coccidiosis is routine and represents a significant production cost.

The development of drug resistance is a serious limitation on the effectiveness of chemotherapy to control the disease. Surveys in the United States, South America and Europe have revealed widespread drug resistance in coccidial protozoa. Since drug resistance is a genetic phenomenon, once established, drug resistance can remain in the population for many years until reduced by natural selection pressure and genetic drift.

The use of drugs in animals used for food production is also coming under increasing scrutiny by the public. Consumers are increasingly concerned with the possibility of drug residues in food. This creates pressure in the poultry industry to reduce the use of drugs to control coccidiosis.

Vaccination of birds against coccidial protozoa is an alternative to chemotherapy. An advantage of vaccination is that it can greatly reduce or eliminate the need to administer anti-coccidial drugs, thus reducing costs to poultry producers, preventing the development of drug-resistant strains, and lessening consumer concerns about drug residues.

Because the immunity conferred is specific to the species of coccidial protozoa used in the vaccine, vaccines must either contain a mixture of species or must contain the species known to be a problem in the particular population of animals to be vaccinated. The use of vaccines containing only species known to be a problem requires that the species in the infective population be identified. Vaccines containing several species are manufactured by combining pure lines of various species of protozoa. Therefore, in order to maintain quality control, it is necessary to periodically examine the lines in order to insure that there has not been contamination by additional species of Coccidia.

Traditionally, species identification of coccidial protozoa has been based on differences in oocyst size and shape, as well as the site of infection in affected animals. Oocyst size alone may not provide the most accurate means of identification, since there is considerable overlap in size between species. The present visual method is slow, expensive and subjective because individual protozoa must be microscopically assessed by personnel trained to visually identify the various species. Efforts have been made to facilitate the visual method by employing computerized image-analysis systems by Kucera et al. (Folia Parasitologica 38: 107-113, 1991), however, such methods still are time consuming and suffered from the inability to satisfactorily separate oocysts in multi-species samples. Also, small particles attached to the oocysts often resulted in false measurements utilizing this system. Therefore, production of coccidial vaccines, either specially manufactured or mixed, would be enhanced by a rapid, accurate and objective means to identify coccidial species.

A relatively recent alternative to identification of cells by microscopic examination has been the use of flow cytometers. While conventional light microscopy is often used to determine qualitative and subjective characteristics of cells or other objects, such as reflected light intensity, flow cytometry allows for the quantitative measure and comparison of light intensity reflected by cells or objects. The most common application of flow cytometry is determining the respective percentages of specific subsets of cells in a population using fluorescently labeled monoclonal antibodies. Using monoclonal antibodies detecting various human antigens, flow cytometry can be used to determine the antigenic profile of a variety of benign and neoplastic cells. (Seminars in Diagnostic Pathology, 6(1): 1-2, 1989). The measurement of intracellular ionized calcium concentrations in living cells using Indo-1, a UV-excitable fluorescent Ca²⁺ indicator, is also a valued application of flow cytometry. (Gilman-Sachs, A., Analytical Chem., 66:700A-707A, 1994). Flow cytometry has also been used in the analysis of hematopoietic cells and the analysis of DNA content for basic research. The term “flow cytometry” is often used synonymously with fluorescence activated cell sorter (FACS).

A typical flow cytometer has several components, including a light source, usually a laser; a sample chamber, flow cell, and sheath fluid stream; a photodetector (used to detect light scatter) and photomultiplier tubes (PMTs) (used to detect that collect light and convert it to electronic signals; a signal processing system that converts analog signals to digital signals; and a computer to direct operations, store the collected signals, and display data. (Gilman-Sachs, supra)

In a typical flow cytometer, particles or cells are directed to flow single file in a stream. Typically the particles or cells are pneumatically driven through tubing where they are injected into a high velocity jet nozzle. The nozzle, containing sheath fluid, typically under pressure, hydrodynamically focuses the particles or cells into a narrow orifice so that the cells exit the nozzle in single file and are constrained to the center of the stream, resulting in laminar coaxial flow. Typical sheath fluids include de-ionized water or a sodium chloride solution, such as Isoton II (BECKMAN COULTER) which may be diluted in, for example, MILLI-Q water. Once the particles or cells exit the nozzle, they flow rapidly through the illumination zone where they pass a light source, commonly an analytical laser. The most commonly used laser is an argon laser, which can emit a very narrow elliptical (cross-section) beam of light ranging from 16×40 μm to 40×700 μm, depending on the beam-shaping optics used in the flow cytometer. (Gilman-Sachs, supra) The most common wavelength utilized is 480 nm, the excitation wavelength of two common fluorochromes. Often, a 15 mW air-cooled laser is sufficient for most analyses. Other types of lasers (He—Ne, Kr, and He—Cd), however, can be used in combination with certain dyes for special applications.

Particles or cells typically pass through the illumination zone at a rate of 1,000 particles or cells per second, although higher rates are possible. As the particles pass through the illumination zone, each particle or cell intercepts the laser beam and scatters some of the laser light in all directions. The incident light is then detected by appropriately placed detectors, such as photocells or PMTs, which measure the magnitude of a pulse representing the extent of light scattered (e.g., the data collected for each cell comprises a “recorded event”). When an object passes in front of the laser beam, the scattered light or the fluorescence emitted from the cell is converted to an electronic signal that is proportional to a specific parameter for that object. The information from a population of cells may be displayed as a frequency histogram on a computer screen. As the light is scattered off of a sporocyst, it scatters in varying directions with varying degrees of intensity. The direction and intensity of the scattered light tells the investigator about the diameter, shape, and granularity of the cell. Using an argon laser, the intensity detection limit of scattered light is typically within about a 37 nanometer wavelength range. The more sophisticated the flow cytometer, the more photodetectors and ultimately more parameters that can be measured for each object.

A typical detection system consists of a forward angle light scatter (FSC) detector and a right angle (90°) side scatter (SSC) detector. Forward light scatter is used to determine particle size. As each cell bisects the laser beam, light from the light source is scattered 360 degrees by the cell. Light scattered in the forward direction (that is, relative to the direction of the laser beam) is roughly proportional to cell size. In the art, it has been empirically shown that light scattered by cells at small angles (0.5-2.0°) in the forward direction is proportional to cell size. Small cells do not scatter as much light as a larger cell. FSC measurements are similar in nature to analyzing a shadow cast by a cell.

Light that is scattered and collected at right angles (that is, light detected at 90 degrees from the axis of the oncoming laser beam (SSC or orthogonally)) is related to cell complexity and cytoplasmic granularity. This measurement is typically used in distinguishing granulated from nongranulated cells. Information obtained from FSC and SSC is referred to as “intrinsic information” as it is based on a cell's size, shape and complexity. Additional information, “extrinsic information,” such as surface antigen or DNA content, can be obtained by the use of appropriate stains, and in particular fluorophores, to stain the organisms. Thus, size, shape, granularity, protein content, DNA content, intracellular pH and calcium concentrations are the most common cellular parameters measured with a flow cytometer. Signals from both FSC and SCC are typically correlated to detect subpopulations of lymphocytes, monocytes and granulocytes in peripheral blood samples.

Flow cytometers further comprise data recording and storage means, such as a computer. Photon emissions collected from photocells and PMT's are converted to analog voltages. These analog signals are then digitized by analog to digital converters (ADCs) and stored on magnetic mediums. Information stored on the magnetic mediums can then be used, with the use of the computer, to construct two- or three-variable histograms, for example, forward light scatter versus side light scatter, or light scattering versus fluorescence intensity. Such histograms can yield information about a variety of properties of interest in identifying the particle. Thus, flow cytometers can rapidly confirm the presence and number of cells in a population and are widely used for that purpose in many fields. See, e.g., the use of a flow cytometer to count blood cells, U.S. Pat. No. 5,627,037, wherein flow cytometry is used to determine an absolute count of one or more populations of cells, such as those sub-populations of blood cells found in unlysed whole blood. To count more than one population of cells, such as CD4⁺ and CD8⁺ lymphocytes, cell markers, such as anti-CD4 and anti-CD8 are required. In general, the use of flow cytometers in microbiology and in particular to identify protozoa has been limited.

Flow cytometry has been used as a means to enumerate protozoa in a sample. Reynolds et al. (J. Appl. Microbiol., 87:804-813, 1999) and Bennett et al. (J. Parasitol. 85:1165-1168, 1999) used flow cytometry to determine the number of one species of oocysts, Cryptosporidium parvum, in samples.

Species specific, fluorescently labeled antibodies have been used in combination with flow cytometry to detect the presence of and quantify infection of cells with Toxoplasma gondii (Gay-Andrieu et al., J. Parasitol., 85:545-549, 1999) and to quantify and detect the presence of a single species of parasite, C. parvum oocysts, for example, in humans (Valdez et al., J. Clin. Microbiol., 35:2013-2017, 1997), horses (Cole et al., J. Clinical Microbiol., 37:457-460, 1999), and mice (Arrowood et al., J. Parasitol., 81(3):404-409, 1995). Flow cytometry has been used to sort C. parvum oocysts from debris before microscopic analysis. (Vesey et al., J. Appl. Bacteriol., 75:87-90, 1993). Flow cytometry has also been used to assess changes in E. tenella sporozoite shape, size, and membrane integrity in response to ionophores (Fuller et al., J. Parasitol., 81:985-988, 1995; Raether et al., Parasitol. Res., 77:386-394, 1991). In addition, flow cytometry, utilizing forward light scatter and fluorescence, has been used to discern between four species of microsporidian parasites of fish (Sparguea lophii, Glugea stephani, Glugea atherinae Berrebi, and Microsporidium ovoideum) and to discern between mono- and diplokaryotic spores of S. lophii. (Amigo et al., J. Euk. Microbiol., 41(3), 1994). Note that in Amigo the process requires nuclei of spores to be dyed.

Fluorescently labeled nucleic acid probes have also been used in combination with flow cytometry to identify protozoa. Vesey et al. (J. Appl. Microbiol., 85:429-440, 1998) used rRNA probes to detect the presence of C. parvum oocysts. Although only a single species of protozoa was used, the authors suggested that species specific rRNA probes could be used to identify species.

Flow cytometry may be used to sort cells. In a flow cytometer equipped to sort cells, as the sheath fluid leaves the nozzle, the nozzle is vibrated at a high frequency, thus breaking the fluid into droplets. Ideally, each droplet contains one cell. When it is determined a droplet contains a desired cell, a charge is applied to the drop. The charged droplet passes through charged plates and is deflected and sorted into a collection vessel.

Flow cytometry has been used to analyze populations of E. tenella oocysts on the basis of size and shape. Fuller and McDougald, (J. Protozool., 36:143-146, 1989), reported that although forward light scatter analysis based on size was useful, it was limited by the considerable overlap in size between species of oocysts as well as the considerable size variation within species. They reported that the combination of size overlap and variation in mixed populations of oocysts, can result in the formation of ambiguous data from which single species cannot be extracted. It was also reported that the comparative analysis of batches of oocysts or identification of species by size is further complicated by the lack of suitable standards for use with flow cytometry. They further report that purification of oocysts by flow cytometry is unlikely to be successful. However, they conclude that flow cytometry may be useful for isolation of large numbers of similarly sized oocysts from mixed field isolates and to isolate single oocysts to obtain absolute purity. Even with such purification, they teach that techniques other than flow cytometry, such as isoenzyme analysis, are needed to confirm species identification.

In all the above instances, either oocysts or coccidial protozoa lacking an outer coat or shell, e.g., sporozoites, were used. The use of oocysts has been shown to produce ambiguous data. Using sporozoites or other coccidial protozoan without an outer coat or shell requires greater care in handling and shortens the period of time in which accurate measurements of identity may be made. Thus, the above-listed techniques do not supply a convenient and accurate method for identification and enumeration of coccidial protozoa and there is a need for a method allowing rapid enumeration and species identification of coccidial protozoa and having a greater degree of certainty than the current art.

Rapid species identification would have several important uses in, for example, the poultry industry. The ability to rapidly and accurately determine which species of coccidial protozoa are causing a disease outbreak would allow veterinarians to determine which of the available anti-coccidial drugs would be the most efficacious. By making use of only the most effective drugs, it is less likely that the protozoa will have sufficient time to develop resistance. In addition, knowledge of the particular species of protozoa present, would allow the administration of vaccines specifically produced to confer immunity against the species known to cause disease in a flock. The method would also be useful for quality control purposes in vaccine production by providing a rapid and accurate means to determine if pure lines of protozoa used to produce vaccines have become contaminated with additional species and if contaminated, to what extent. The present invention addresses all of these needs.

### SUMMARY

The present inventor has surprisingly found that by using sporocysts it is possible to quickly and accurately identify, enumerate, and to sort mixed populations of coccidial protozoa by species without having to use specific antibodies or probes. Thus the present invention provides a simple, rapid method for sorting and identification of coccidial protozoa in a mixed population.

This invention relates to a method for the identification of coccidial protozoa in a sample by the use of flow cytometer. The sample of protozoa used can be obtained from either a naturally occurring population or from a population of protozoa maintained in a laboratory. In the invention, a sample of protozoa at the sporocyst stage is provided. Sporocysts can be naturally occurring or can be produced from oocysts using well known methods. The sporocysts are then analyzed by flow cytometry and sorted on the basis of size (utilizing forward light scatter) and granularity (utilizing side light scatter) which are used to define data output regions characteristic of a species of protozoa. It has been determined that sporocysts, unlike other life stages of coccidial protozoa, provide, when analyzed using a flow cytometer under the conditions set forth herein, a reliable sample of traits that allows one to quickly and accurately identify, enumerate, and to sort mixed populations of coccidial protozoa by species without the use of specific antibodies or probes. In a preferred embodiment, FSC and SSC characterize the parameters used to identify a specie or species of coccidial protozoa.

One aspect of the invention provides, therefore, a method for determining the number and species of coccidial protozoa in a sample comprising providing a sample of sporocysts of said protozoa; defining data output regions of a flow cytometer based on a measurement selected from the group consisting of forward light scatter, side light scatter, and fluorescence; analyzing the sporocysts of said sample using a flow cytometer; and determining the number or percentage of each species of coccidial protozoa in the sample.

Another aspect of the invention includes a method for determining the species and number of protozoa of the genus Eimeria comprising defining data output regions of a flow cytometer based on forward light scatter and side light scatter, said data output regions characterized as containing at least about 85% of the recorded sporocysts of the species of interest; providing a sample of sporocysts from a population; analyzing said sample using said flow cytometer; and determining the number of each species of coccidial protozoa in the sample.

Yet another aspect provides a method for determining the number and species of coccidial protozoa in an environmental sample comprising defining data output regions of a flow cytometer based on forward light scatter and side light scatter, said data output region characterized as containing at least 50% of the recorded sporocysts of the species of interest; isolating the protozoa from the sample; if the protozoa are at the oocyst stage, inducing sporulation and isolating the sporocysts; analyzing the sporocysts using a flow cytometer; and determining the number or percentage of each species of coccidial protozoa in the sample.

Yet another aspect provides a method for determining the number and species of coccidial protozoa of the genus Eimeria in an environmental sample comprising defining data output regions of a flow cytometer based on forward light scatter and side light scatter, said data output region characterized as containing at least 85% of the recorded sporocysts of the species of interest; isolating the protozoa from the sample; if the protozoa are at the oocyst stage, inducing sporulation and isolating the sporocysts; analyzing the sporocysts using a flow cytometer; and determining the number or percentage of each species of coccidial protozoa in the sample.

Still another aspect provides a method for treating an animal having a coccidial infection comprising obtaining a sample from the animal, a representative animal in a group, or the environment in which the animal is housed; isolating the coccidial protozoa from the sample; if the protozoa are at the oocyst stage, inducing sporulation and isolating the sporocysts; defining data output regions of a flow cytometer based on forward light scatter and side light scatter, said data output regions characterized as containing at least 50% of the recorded sporocysts of the species of interest; analyzing the sporocysts using a flow cytometer; and treating the animal with an anti-coccidial drug or drugs effective against the species of coccidial protozoa identified.

A method for treating an animal infected with protozoa of the genus Eimeria comprising obtaining a sample from the animal, a representative animal, or the environment in which the animal is housed; isolating the protozoa from the sample; if the protozoa are at the oocyst stage, inducing sporulation and isolating the sporocysts; defining data output regions of a flow cytometer based on forward light scatter and side light scatter, said data output regions characterized as containing at least about 85% of the recorded sporocysts of the species of interest; analyzing said sample using said flow cytometer; and treating the animal for the species of protozoa identified.

And yet another aspect provides a method for determining the number and species of coccidial protozoa in a pharmaceutical composition comprising, obtaining a sample of the composition; if the protozoa in the sample are at the oocyst stage, inducing sporulation and isolating the sporocysts; defining data output regions of a flow cytometer based on forward light scatter and side light scatter, said data output region characterized as containing at least 50% of the recorded sporocysts of the species of interest; analyzing the sporocysts using a flow cytometer; determining the number or percentage of each species of coccidial protozoa in the sample; and calculating the number of each species in the pharmaceutical composition.

The invention is further directed to a method for determining the species of coccidial protozoa in a sample. The method comprises passing an analyte comprising a suspension of unknown sporocysts of said protozoa sample in a liquid medium through a flow cytometer; impinging light on said analyte passing through the flow cytometer; and measuring at least one characteristic of said sample. The characteristics may be selected from the group consisting of forward flow light scatter, side light scatter, and fluorescence. A range of values obtained from measurements is used to determine a pattern of measurements obtained from said analyte with respect to at least one of said characteristics. One then compares said pattern of measurements for said analyte with at least one reference pattern of measurements with respect to at least one characteristic as obtained when at least one reference suspension of known sporocysts is passed through said flow cytometer, and correlating the reference pattern for the analyte with at least one reference pattern of measurements, to determine the species of protozoa in said analyte.

The invention is further directed to a method wherein patterns of measurements determined for an analyte and patterns of measurements determined for a reference suspension constitute a plurality of data output regions from a flow cytometer. All data output regions being within a field having the dimensions of a plurality of characteristics. When at least 85% of the data measured by the flow cytometer for said plurality of characteristics of said analyte is contained within said plurality of reference data output regions, said plurality of data output regions for said analyte indicates said analyte comprises sporocysts of an identity correlating to said known identity of said plurality of said data output regions.

The invention further comprises a method for determining the species of coccidial protozoa in a sample comprising passing an analyte comprising a suspension of unknown sporocysts of said protozoa sample in a liquid medium through a flow cytometer; impinging light on said analyte passing through said flow cytometer; measuring at least one characteristic of said sample. The at least one characteristic being selected from the group consisting of forward flow light scatter, side light scatter, and fluorescence. One then determines a pattern of measurements obtained from said analyte with respect to said at least one characteristic and then compares said pattern of measurements for said analyte with a reference pattern of measurements with respect to said at least one characteristic as derived from measurements taken in passing known sporocysts through a flow cytometer and determining the species of protozoa in said analyte.

### BRIEF DESCRIPTION OF THE DRAWINGS

These and other features, aspects, and advantages of the present invention will become better understood with regard to the following description, appended claims and accompanying figures where:

FIG. 1 shows the results of analysis of E. acervulina sporocysts by flow cytometer. The perimeter identified as R1 is the data output region characteristic of E. acervulina sporocysts.

FIG. 2 shows the results of analysis of E. maxima sporocysts by flow cytometer. The perimeter identified as R3 is the data output region characteristic of E. maxima sporocysts.

FIG. 3 shows the results of analysis of E. tenella sporocysts by flow cytometer. The perimeter identified as R4 is the data output region characteristic of E. tenella sporocysts.

FIG. 4 shows the results of analysis of a mixed population of E. acervulina, E. maxima and E. tenella sporocysts by flow cytometer. The perimeters identified as R1, R3 and R4 are the data output regions characteristic of E. acervulina, E. maxima and E. tenella sporocysts, respectively. Region R2 is used for calibration.

In all figures units are arbitrary.

### DEFINITIONS

FSC=forward light scatter

SSC=side light scatter

PBS=phosphate buffered saline (8.0 g NaCl, 0.2 g KH₂PO₄, 0.2 g KCl, 1.15 g Na₂HPO₄ in 1 L H₂O, pH 7.2)

“Animal” means any vertebrate animal and includes human beings.

“Analyte” means a composition containing sporocysts of an unknown species and/or purity. This is in contrast to a reference suspension or reference sample.

“Sample” means a representative portion of a whole, but can include the whole. Thus, for example, a sample can be a representative portion of a composition but also the entire composition.

“Data output region” comprises a pattern of measurements wherein said pattern comprises a frequency distribution of combinations of measurements, such as forward light scatter, side light scatter and fluorescence, obtained by analyzing a sporocyst-containing analyte or reference suspension and characterized by a geometric perimeter.

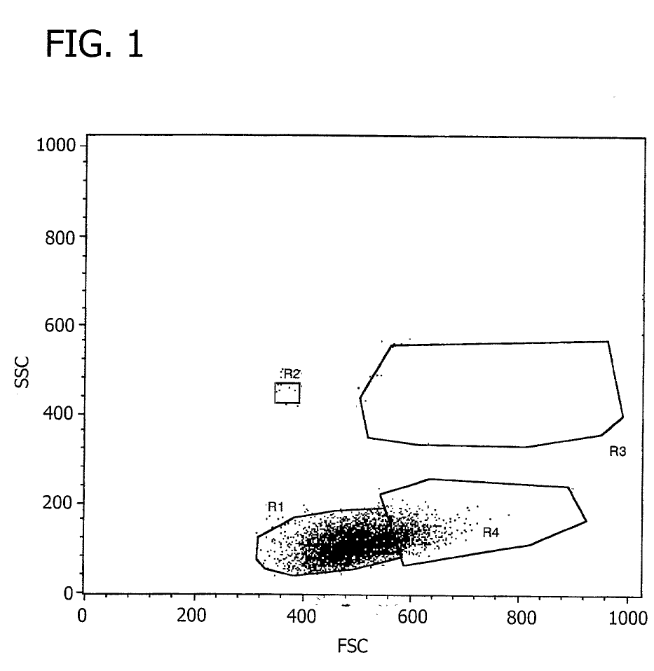

“Frequency distribution” comprises the number of occurrences of a data value in an analyte or reference suspension. Frequency distribution may also comprise the number of values falling within a fixed range. Data can be summarized in this manner by tabulating all the data values into distinct ranges and then counting the number of times data values within each range appears in the frequency distribution. An example of two-dimensional frequency distributions are seen in FIGS. 1-4. A graphical representation, such as a bar chart or histogram, may also be used to represent a frequency distribution.

“Histogram” comprises a graphical display showing the distribution of recorded sporocysts in a sample by dividing the range of the data into non-overlapping intervals and counting the number of values which fall into each interval.

“Oocyst” means the dormant life-cycle stage of a coccidial protozoan having a tough outer coat. As formed, the oocyst is not capable of infection and may also be referred to as an unsporulated oocyst. Oocysts are found in the intestine of animals following release from infected cells and are eliminated in the excreta.

“Reference suspension” may comprise a suspension containing one known species of sporocysts, a suspension containing reference spheres of a purported size, or a suspension containing multiple known species of sporocysts in known concentrations. Also referred to herein as a reference sample.

“Sporulated oocyst” refers to an oocyst which has undergone maturation naturally or through artificial manipulation such that the sporulated oocyst is capable of infecting a susceptible host. During maturation, a multiplicity of sporocysts, each with a membrane and outer shell or case, develops within the oocyst creating a “cyst within a cyst.” For example, in E. tenella, a sporulated oocyst contains four sporocysts, each with their own outer shell or case.

“Sporocyst” refers to a life-cycle stage of a coccidial protozoan having an outer coat or case containing a multiplicity of sporozoites which are the ultimate infective agent of the protozoan. In the instance of E. tenella, each sporocyst contains two sporozoites.

“Encysted protozoa”, “encysted oocyst” and “encysted sporocyst” all refer to organisms which are within a cyst or have their own outer coat or shell.

“Excysted protozoa” and “excysted sporozoite” both refer to an organism that has naturally or mechanically been released from a cyst. Thus, as defined herein, a sporocyst is not considered to be an excysted protozoan; a sporocyst is usually mechanically excysted from an oocyst. A sporozoite is usually enzymatically released from a sporocyst.

“Recorded sporocyst” refers to a sporocyst that has been detected and analyzed by a flow cytometer.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

The sample from which the coccidial protozoa are obtained can come from a variety of sources. In one embodiment, the sample is obtained from the animal to be treated or vaccinated. Alternatively, the sample is obtained from a representative animal or sample of animals within a large group of animals such as a herd or flock. The sample may comprise feces, tissue samples or a combination of both. An example of a suitable tissue sample is scrapings from the digestive tract and in particular the intestinal lining of animals. Although fecal samples can be obtained directly from animals, fecal samples may also be collected from the environment in which the animals are housed, for example, from bedding or litter. Samples may also be obtained from other sources in the environment in which animals are housed such as bedding or litter, soil, water, feed, or equipment.

Once a suitable sample(s) is obtained, the protozoa are isolated. Several methods for isolating protozoa are known in the art and can be used in practicing the present invention. A review of several methods for the isolation of oocysts can be found in Ryler et al. (Parasitology 73:311-326, 1976). In one method, described in U.S. Pat. No. 3,147,186, oocysts are isolated by mixing the material with a liquid and allowing the heavier particles to settle to the bottom away from the lighter oocysts. The upper layer containing the oocysts is then removed. Another method involves the use of solutions of sufficient specific gravity, typically 1.2, so that the oocysts float to the top of the solution. Generally these solutions are made up of water to which a sugar (e.g. sucrose), ZnSO₄, or NaCl has been added to increase the specific gravity to the desired value. In the flotation method, a preliminary step of filtering the material through a sieve or cheesecloth to remove large particles is often included. After mixing with the solution, the oocyst mixture is centrifuged and the oocysts removed from the surface layer. The centrifugation step may be repeated several times.

Another method involves gradient centrifugation. The gradient used can be discontinuous or continuous. An example of a typical gradient for coccidial oocysts is 0-50% sucrose. In this method the material containing the oocysts is placed on top of the gradient and centrifuged. Following centrifugation, the layer containing the oocysts is recovered. The process may be repeated in order to increase the purity of the resulting oocyst preparation. As with flotation, this method is preceded by filtration of the material.

Additional methods of oocyst isolation include, the use of glass bead columns (Ryler et al., Parasitology, 73:311-326, 1976) and the bicarbonate ether method (Smith and Ruff, Poultry Sci. 54:2081-2086, 1975). In the glass column method, the aqueous suspension of fecal matter is added to a mixture of glass beads and a detergent, for example 5% Tween 80. The mixture is then applied to a column of glass beads and the oocysts are allowed to flow through while much of the undesired fecal matter is retained in the column. The effluent may then be concentrated by centrifugation.

In the bicarbonate ether method, the feces from infected chickens is strained, through cheese cloth for example, and the liquid fraction is captured while the solid fraction is discarded. The liquid fraction is then concentrated by centrifugation. The solid fraction is recovered and the supernatant is discarded. The recovered solid fraction is then resuspended in a solution of 1% sodium bicarbonate. To the resuspended solid fraction, now in suspension, is then added ether in a volume approximately equal to the volume of 1% solution of sodium bicarbonate. The mixture is then centrifuged. The debris plug and supernatant is discarded while the sediment is washed by resuspension in water. This suspension is then centrifuged and the supernatant discarded. The sediment is then recovered for use. (Smith and Ruff, Poultry Sci. 54:2081-2086, 1975).

Yet another method involves a combination of sieving, continuous centrifugation and filtration. In this method preliminary purification is achieved by sieving the material through sieves having progressively smaller openings. Further purification is achieved by continuous centrifugation using a solution having a specific gravity preferably 1.01 to 1.2 g/l. As a further purification step, the material from the centrifugation containing the oocyst is filtered using a membrane of a pore size that retains the oocysts.

For example, if the sample consists of feces, the excreta can be mixed with a minimum of 2 volumes (w/v) of saturated aqueous NaCl to form a slurry. If necessary, the slurry can be processed in a mixer or blender until a homogenous consistency is achieved. The slurry is centrifuged at about 800×g for 10 minutes at 4° C. The supernatant is collected by pouring through a double layer of 24×24 weave cheese cloth. The filtered supernatant is diluted with two volumes of potable water and centrifuged at about 1600×g for 10 minutes at 4° C. The pelleted protozoa, typically at the oocyst stage, are washed with water and pelleted by centrifugation at about 1600×g for 10 minutes at 4° C. an additional three times. The protozoa are then washed three times in 2.5% potassium dichromate using the same procedure used for the water washes. After the final wash, the protozoa can be stored in 2.5% potassium dichromate at 4° C. or transferred to a container for sporulation.

Alternatively, sequential filtration can be used to isolate protozoa based on size. In an alternative procedure, the excreta is placed in a container at about 40-50° F. and mixed with water, preferably at a ratio of about 2 pounds of excreta per gallon of water. The mixture is agitated to form a slurry and then pumped into a vibrating sieve fitted with screens, preferably of between 50 to 250 mesh, to remove the large particulate matter. The material passing through the sieve is collected and pumped into a continuous flow centrifuge also maintained at about 40-50° F. The resulting centrate is discarded while the solid material containing the oocysts is collected and added to an equal volume of concentrated sucrose or high fructose corn syrup (HFCS). To this is added an equal volume of water for a total final volume of about four time the volume of the initial solids. The specific gravity of the final mixture is preferably sufficient to float the oocysts, preferably between about 1.01 to 1.2 g/l and more preferably about 1.09 g/l. The final mixture is then pumped into a continuous centrifuge at a rate to allow the oocysts to remain in the centrate. The solids are discarded. Concentration of the oocysts and removal of the residual sugar can be accomplished by continuous flow centrifugation at a high feed rate which allows the more buoyant oocysts to float on a homogenous solution of water and sucrose. Alternatively, the material can be centrifuged in a rotor centrifuge. In this case, the supernatant is discarded and the oocysts in the resulting pellet are resuspended in water. Additionally, the residual sugar can be removed by filtration using filters with a pore size which excludes the oocysts. When filtration is used, tangential flow is preferred. The isolated oocysts are placed in sterile water or 2.5% potassium dichromate and stored at 2-8° C. until sporulation.

Typically, the coccidial protozoa in the sample obtained will be at the oocyst stage of the life cycle. In such cases, the oocysts are induced to undergo sporulation. Methods for sporulation of coccidial oocysts are well known in the art.

Sporulation is an aerobic process that occurs between 1 to 43° C., preferably between 15 to 38° C. more preferably between 20 to 30° C. and more preferably still between about 28 to 30° C. Sporulation systems can be either static or used forced aeration. In static systems, the liquid containing the oocysts is held in an atmosphere of air or oxygen. In forced aeration systems air or oxygen is bubbled through the sporulation liquid. Alternatively aeration is achieve by mixing the liquid either by shaking, swirling or stirring. Typically, sporulation solutions also contain between 2 to 3% potassium dichromate.

For example, sporulation can be accomplished by placing oocysts in an aqueous solution of 2.5% potassium dichromate in a container and placing a cotton or foam rubber plug in the opening. The container is agitated on an orbital shaker at 250 rpm and 28-30° C. for 48-72 hours. Alternatively, the solution containing the oocysts can be constantly agitated while air is bubbled through the solution at about 25 to 75 cubic feet per hour. During the sporulation process, samples are withdrawn at regular intervals and tested for sporulation by microscopic examination of the oocysts to determine if they contain sporocysts.

Following sporulation, sporulated oocysts may be collected by filtration, centrifugation or other acceptable concentration procedures known to those skilled in the art. For example, sporulated oocysts can be concentrated by centrifugation at about 1600×g for 10 minutes at 4° C. and the supernatant discarded. After concentration, sporulated oocysts may be disinfected, for example, by suspension in 5.25% sodium hypochlorite for 10 minutes at 4° C. Following disinfection, the sporulated oocysts are removed from the sodium hypochlorite by dilution and centrifugation at 1600×g for 10 minutes at 4° C. or other acceptable means. The sporulated oocysts are then washed four to six times with sterile water. After washing, the sporulated oocysts can be stored in 2.5% potassium dichromate, phosphate buffered saline (PBS) containing 30 μg/ml gentamicin or other acceptable disinfectants or preservatives.

Following sporulation, sporocysts are obtained from the sporulated oocysts. Various methods for isolating sporocysts from sporulated oocysts are well known to those of ordinary skill in the art and include mechanical, chemical and enzymatic disruption of the oocyst wall. By way of a representative example, a method of mechanical disruption will be described herein. Sporulated oocysts are diluted to a concentration of about 2×10⁶/ml in PBS. One ml of the 2×10⁶/ml suspension of sporulated oocysts is added to 1.5 ml microfuge tubes containing 125 μl of 0.5 mm glass beads. The tubes containing the sporulated oocysts and the glass beads are vortexed at high speed for five minutes and then chilled on ice. Following vortexing, a 10 μl sample can be examined under the microscope to determine the extent of oocyst disruption. If a significant number of oocyst remain intact, the sample may be repeatedly vortexed for one minute intervals until virtually all of the oocysts have been disrupted.

Following vortexing, the supernatant is removed and saved. The glass beads are then washed with PBS and the supernatant saved and combined with the supernatant obtained after vortexing. The supernatant is then centrifuged at about 9020×g for about two minutes for samples containing E. maxima or E. tenella and three minutes for samples containing E. acervulina or mixed populations. The supernatant is discarded. The pellet is resuspended in 0.2 ml of PBS. Although the pellet will preferably contain primarily sporocysts, it will be apparent to those skilled in the art that intact oocysts, empty oocyst coats, and sporozoites may also be present. It is preferred that the preparation be further purified to increase the proportion of sporocysts present using methods commonly known in the art. For example, and without limitation, sporocyst isolation can be accomplished by centrifugation using 50% Percoll as described by Dulski and Turner (Avian Diseases 32:235-239, 1988), herein incorporated by reference. Briefly, the pellet obtained from directly above is resuspended in 1 ml of 50% Percoll, centrifuged as above and the supernatant discarded. The resulting pellet is again suspended in 50% Percoll, centrifuged and the supernatant discarded. The final pellet is resuspended in PBS. The concentration of protozoa per ml is then determined by using a hemacytometer or other method of determining cell number.

The number of sporocysts in each sample to be analyzed will vary with the flow cytometer used. Preferably the sample will be large enough to provide reliable and repeatable identification of the coccidial protozoa of interest. Although it may be necessary to test several sample sizes to determine the optimum sample size, such testing is routine in the art and can be accomplished by the skilled technician without undue experimentation.

The final concentration of the sample may vary and such final concentration is limited by the capacity of the flow cytometer used with the instant invention. A dilute sample will take longer to analyze as a greater elapsed time expires in order to obtain a reliable set of data points. A sample that is overly concentrated cannot be analyzed with accuracy by some flow cytometers.

In one embodiment, for each sample to be analyzed by flow cytometry, about 80,000 sporocysts are analyzed. The sporocysts are placed in a container and diluted with PBS. Preferably, the container is compatible with the flow cytometer to be used. In one embodiment, the sporocysts are placed in polystyrene tubes, for example, 12×75 mm (FISHER No. 14-961010) tubes, and PBS added to 600 μl. Thus, the final sample comprises about 80,000 sporocysts per 600 μl. Such concentrations have been found to produce reliable data in convenient periods of time.

Optionally, the sporocysts can be stained with a dye or stain to aid in analysis. Any dye or stain capable of staining coccidial sporocysts and known in the art can be used. In one preferred embodiment, the stain or dye is a fluorescent stain or dye. A non-limiting example of a suitable dye is ethidium bromide which can be used at a concentration of between 2 μg/ml and 10 μg/ml to stain sporocysts.

The sporocyst sample is analyzed using a flow cytometer. The sporocyst sample, that is, the suspension containing one or more unknown species of sporocysts, in particular from the genus Eimeria, is the “analyte” and is thus analyzed using a flow cytometer. The analyte may also comprise commercially available coccidiosis vaccines wherein a consumer desires to evaluate and assess purity and species content of that vaccine. Regardless of the source of the analyte, the analyte can be diluted or concentrated, depending upon initial concentration, to a concentration that is acceptable for use with the flow cytometer being used in connection with the instant invention. Preferably, the analyte contains 80,000 sporocysts per 600 μl.

Flow cytometers that may be used in connection with this invention include, but are not limited to, a Becton-Dickinson FACScan (Becton-Dickinson Corp., Mountain View, Calif.), or its equivalent. The flow cytometer useful in the method of the instant invention may be utilized in association with software that facilitates analysis of data generated by the flow cytometer. Software that may be used includes, but is not limited to, CellQuest software (Becton-Dickinson Corp.). Software is used to analyze the frequency distribution of reference suspensions to facilitate the creation of reference data output regions. Software is also used to analyze frequency distribution data generated by analytes. Software that can discern the percentage of analyte data that occurs in a reference data output regions is preferred. The analyte, comprising a suspension of unknown or putatively known sporocysts of a sample of protozoans in a liquid medium, is passed through a flow cytometer. A light source, typically an argon laser, is used to impinge light on the analyte flowing through the flow cytometer.

The flow cytometer is adjusted to measure at least one characteristic of the analyte, for example, the flow cytometer may be adjusted to collect data on both forward light scatter (FSC) and side light scatter (SSC). If a fluorescent dye is used, the flow cytometer is also set to record the intensity of light emissions at the wavelength characteristic of the dye or dyes used. Fluorescence intensity may be measured using either a log scale or a linear scale. When measuring bright fluorescence a linear scale is often used. When measuring a dim fluorescence, a log scale is typically used.

Prior to the analysis of unknown samples, an instrument is calibrated. Calibration can be accomplished by the use of reference spheres of known size (such as COULTER Check Fluorosphere Beads) (Coulter Corp., Hialeah, Fla.), light scatter, and, if applicable, fluorescent characteristics and/or by the use of single species populations of sporocysts (reference suspension). Data obtained from calibration samples can be used to define reference data output regions, that is, a frequency distribution of combinations of measurements, such as forward light scatter, side light scatter, and fluorescence, obtained by analyzing a reference suspension containing reference spheres or known specie(s) of sporocyst. Any combination of FSC, SSC and/or fluorescence which allows identification of the coccidial species of interest can be used. Preferably, reference suspensions used to calibrate an instrument or used to create reference data output regions are at concentrations substantially similar to the concentration of the sample to be analyzed. More preferably, the reference suspension and the analyte are at the same concentration. Most preferably, the concentration of the reference suspension and the analyte is 80,000 sporocysts per 600 μl.

Data from a sample containing sporocysts of an unknown specie or species, once analyzed by a flow cytometer, may then be processed, for example, by utilizing the appropriate acquisition and analysis software. Software, such as CellQuest, facilitates acquisition of data, that is, the software processes and stores data from the analysis of an analyte. The software can be used to store the settings of the instrument used (both when analyzing analytes and when analyzing reference suspensions). Settings that are of note include the voltage utilized as a difference in voltage will greatly alter results. Such software can also be used for subsequent analysis, that is, the comparison of the acquired data to reference data output regions. Such software can store a data pattern generated by the measurement of at least one characteristic of the sample. The characteristic may be selected from the group consisting of forward flow light scatter, side light scatter, and fluorescence. It has been determined that different species of sporocysts register different patterns based on measurements of one or more of the above-listed characteristics. The pattern, or data output region, generated by a sample containing an unknown specie or species of protozoa may then be compared to reference patterns, e.g., reference data output regions, to determine the specie or species present in an analyte. The reference pattern comprises a known frequency distribution with respect to at least one characteristic for known sporocysts or at least one reference suspension over the same range of values as obtained when known sporocysts or at least one reference suspension is passed through a flow cytometer under reference conditions.

Data analysis software performs this comparison by comparing the frequency distribution for at least one of the above-listed characteristics, preferably the frequency distribution for more than one characteristic, with the pattern of measurements of the same characteristic or combination of characteristics as generated from the analyte. Software can provide quick and accurate quantitative data analysis that provides, for example, the percent of the frequency distribution for an analyte being within a reference data output region.

Flow cytometers can analyze a large number of protozoa over a short course of time. Some flow cytometers can analyze up to 10,000 cells per second. Typically a histogram, involving at least one parameter is displayed in real time, i.e., while the sample is running through the flow cytometer. It is possible to quantify the number of sporocysts in a sample by the use of various computer programs. Such programs can rapidly count the number or recorded sporocysts in a sample. The number of recorded events is equal to the number of sporocysts in a sample. In particular, it is possible to quantify the number of sporocysts that are positive for a particular parameter in mixed species population. Software can also be used to count the number of recorded sporocysts whose frequency distribution falls within a reference data output region.

Reference suspensions typically comprise the same medium, or the equivalent, as an analyte. In one embodiment the medium comprises 1×PBS. The medium used may vary but it is preferred that the mediums for the reference suspension and the analyte are substantially the same.

A reference pattern, or data output region, is obtained by passing the reference suspension or known sporocysts through a flow cytometer and impinging light on said suspension under conditions that are substantially the same as conditions used when analyzing an unknown sample. Such conditions preferably include substantially the same sheath fluid, flow rate, temperature, wavelength of the laser, power setting of the laser, and, as mentioned above, measurements of at least one of the same characteristics (FSC, SSC, and/or fluorescence). In one embodiment, the same flow cytometer and conditions are used with respect to both the reference suspension and the unknown sample. In another embodiment known sporocysts are passed through a flow cytometer to determine a reference pattern of measurements used to draw a perimeter of a reference pattern. In a preferred embodiment, a plurality of suspensions containing known sporocysts are passed through a flow cytometer to determine a plurality of patterns of measurements that are compiled to form a composite pattern of measurements that define a reference pattern of measurements.

The perimeters of a reference data output region define a region where a pattern of measurement for at least one characteristic is associated with a particular species. For example, in FIG. 1, R1 defines the reference data output region for E. acervulina. When data is generated from an analyte, and at least 50% of said data is located within a data output region for a known species, said analyte is thus determined to contain the known species. Certainty of identification is enhanced when at least 85% of the data generated from an analyte falls within a data output region for a known species. Greater certainty is assured when at least 90% of the data generated from an analyte falls within a data output region for a known species. For quality control purposes, it may be preferable to establish as a criterion the requirement that at least 95% of the data generated from an analyte fall within a data output region for a known species.

In another aspect of the invention, multiple reference suspensions, all containing the same specie or species, are analyzed to create multiple reference data output regions. The gross data is further analyzed to establish a well-defined reference data output region that is more representative of the known specie or species. In another aspect of the invention, a reference pattern is generated by compiling a plurality of pattern of measurements, generated by either suspensions containing known oocysts or reference suspensions, to create a composite pattern of measurements. Compilation of multiple data output regions for a known specie or species to create a composite pattern of measurements is preferred as background debris and intact oocysts not removed during sporocyst purification may alter the perimeter of any one pattern of measurements. In addition, and without being bound by any one theory, inconsistencies in reference data is likely due to variations in the buffer used to suspend the sporocysts. Furthermore, the parameters for flow cytometry operation, i.e., the characteristics measured, and the perimeters for reference data output regions may be stored in data files that can be transferred to a subsequent flow cytometer having compatible software.

Patterns of measurements for an analyte comprise the frequency distribution of measurements over a plurality of ranges of values for at least one of the above-mentioned characteristics. Once obtained, the patterns of measurements of an analyte are compared to the pattern of measurement of at least one reference sample. Such comparison is preferably over the same plurality of ranges of values as obtained when the reference suspension is passed through a flow cytometer under the reference conditions. More preferably, the pattern of measurements obtained from an analyte is compared with a plurality of patterns of measurements from reference suspensions, wherein the reference suspensions contain sporocysts of different species. In one embodiment, the pattern of measurements, or data output region, for an environmental sample that may contain one or more species of coccidial protozoa from the genus Eimeria is compared to reference patterns that contain patterns of measurements from reference suspensions containing coccidial protozoa from the genus Eimeria, for example, the reference suspension may contain sporocysts from E. tenella, E. acervulina, and E. maxima. For example, the pattern of measurements from an environmental sample can be compared to data output regions R1 through R4 as set forth in any one of FIGS. 1 through 4.

A pattern of measurements, as used herein, is made utilizing a flow cytometer and preferably comprises combinations of measurements of a plurality of different characteristics of an analyte. The pattern of measurements determined for an analyte comprises a frequency distribution of measured values of a characteristic, or a frequency distribution of combinations of measured values of a plurality of characteristics. In a histogram, the frequency distribution may be expressed by the number of measurements of a characteristic that fall within each of a plurality of successive ranges of values, or which fall within each of a plurality of contiguous combinations of ranges of values. A reference pattern consists of a frequency distribution of measured values or combinations of measured values for the same characteristic or combination of characteristics that are measured in the analyte, all obtained under reference conditions; and when expressed in a histogram is preferably taken over the same ranges of values or combinations of ranges of values used to construct the histogram for the analyte. Preferably, the analyte pattern is determined under the same conditions used to establish the reference pattern.

A pattern of measurements determined for an analyte or for a reference suspension constitutes a data output region of a flow cytometer. Data output regions can be generated by analyzing reference suspensions and analytes containing unknown numbers and species of coccidial protozoa. Data output regions fall within a field having the dimensions of the plurality of characteristics assessed. Certainty of identification is enhanced when at least about 85% the data generated by an analyte is characterized as contained within the data output region generated by the flow cytometer for a plurality of characteristics of a reference suspension.

Typically, the data obtained are displayed as two or three dimensional histograms depending on the number of parameters measured. Areas or peaks within the histograms can then be identified which are characteristic of the species of interest. Such histograms reflects the frequency of at least two of a plurality of characteristics measured, that is; FSC, SSC, or fluorescence. If more than one species is present in the sample, statistical analysis of the data can be used to determine the proportion of each species within the sample.

If within its capabilities, the flow cytometer can also be used to sort the protozoa within the sample on the basis of species using the data parameters determined to be characteristic of the species.

In a preferred embodiment, data obtained are used to create two dimensional histograms of SSC vs. FSC. Such histograms show patterns of measurements that comprise a frequency distribution of a plurality of combinations of such measurements over a plurality of combinations of ranges of values for the plurality of combinations of measurements.

In one embodiment, using data from the reference spheres or reference suspensions, data output regions are defined by drawing perimeters around areas on the SSC vs FSC histograms which contain events for the species of interest. Such perimeter may be, for example, drawn manually on a print out of the histogram showing a pattern of measurements for a reference suspension. In one embodiment an acetate sheet, or other translucent material, may be successively laid over a plurality of patterns of measurements comprising the frequency distribution of measurements over a range of values for at least one characteristic and perimeters are drawn thereon for each successive reference pattern of measurements to compile multiple reference data output regions. The multiple reference patterns are compiled to create a composite pattern of measurements. The perimeter of a composite reference pattern of measurements is drawn to capture a majority of the frequency distribution generated by a plurality of known sporocysts. In yet another embodiment, the perimeter of a composite reference pattern of measurements is drawn to capture a majority of the frequency distribution generated by a plurality reference suspensions. Alternatively, one may utilize software to draw such perimeters. Parameters used to draw the perimeter can include, but is not limited to, the mean and/or mode of the frequency distribution generated by a reference suspension.

In one embodiment, the perimeter drawn captures about 85% of the recorded events. Preferably, the perimeter captures about 90% of the recorded events. One skilled in the art will recognize that some recorded events are indicative of debris or contamination in the reference suspension and will not consider such outliers in determining the placement of the perimeter. Regardless of whether the perimeter was drawn manually or by utilizing computer assistance, the geometric perimeter is then saved on a computer utilizing software. In a preferred embodiment, a composite perimeter is defined by successively comparing perimeters drawn around data generated from multiple reference suspensions containing the same known species of coccidial protozoa. In one embodiment, a composite perimeter is derived by analyzing an acetate upon which multiple reference data output region perimeters have been drawn and eliminating irregularities and differences between and among each reference data output regions. Software may also be used to compile and analyze multiple reference data output regions to derive a composite data output region. Such software can eliminate irregularities and differences between and among a plurality of perimeters and/or determine the mean and/or mode of a plurality of perimeters. Composite perimeters define a reference pattern of measurements, also referred to herein as a reference data output region.

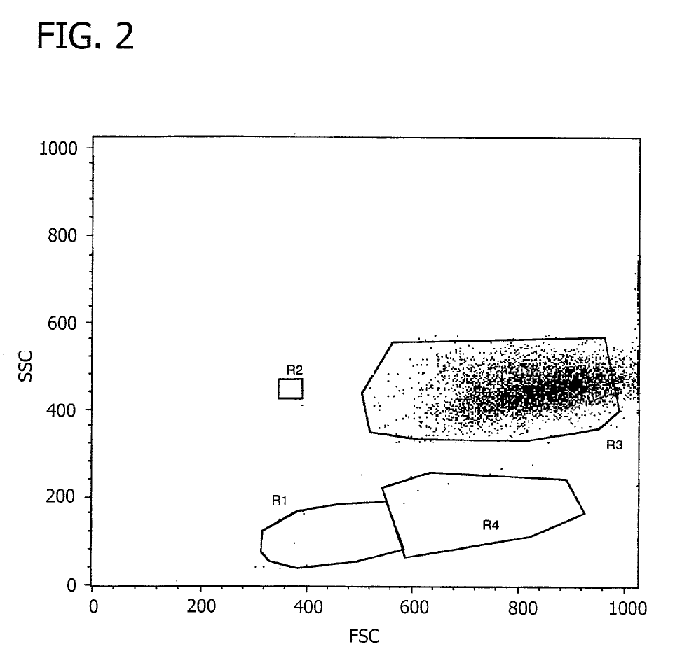

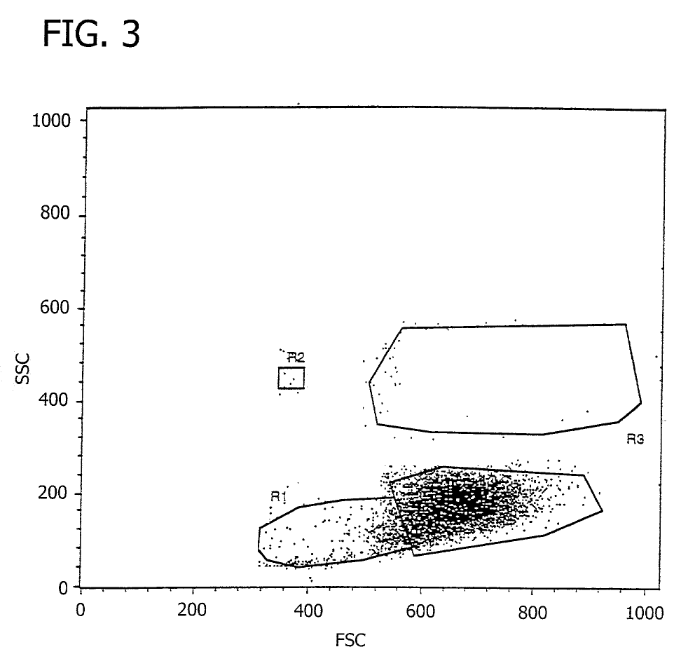

Although the data output region can be defined based on the results of a single analysis of reference spheres or reference suspension, it is preferred that the data output region be based on a composite of several analyses. In one embodiment, the data output region is defined by a composite of at least three analyses of a reference suspension wherein said reference suspension contains from about 2500 to about 5000 counted sporocysts per assay before cessation of analysis. In yet another embodiment, the data output region perimeter is defined by a composite of at least 10 analyses, in another embodiment by a composite of at least 20 analyses, and in still another embodiment by a composite of at least 30 analyses. FIG. 1 shows an analysis of E. acervulina with the perimeter that defines a composite data output region for E. acervulina being R1. FIG. 2 shows an analysis of E. maxima with the perimeter that defines a composite data output region for E. maxima being R3. FIG. 3 shows an analysis of E. tenella with the perimeter that defines a composite data output region for such species as being R4. Region R2 was used for calibration purposes. If more than one species of sporocyst is present, the percentage of each species can be calculated by dividing the number of sporocyst in one region by the total number of sporocyst for all regions containing sporocysts.

In yet another preferred embodiment, the sporocyst-containing sample analyzed comprises from about 170 sporocysts to about 40 sporocysts per μg. In a more preferred embodiment, the sporocyst-containing sample comprises from about 150 sporocysts to about 115 sporocysts per μg. In a most preferred embodiment, the sporocyst-containing sample comprises about 130 sporocysts per μg. Such concentration may be obtained by suspending approximately 80,000 sporocysts in 600 μg of PBS. This results in 130,000 sporocysts per milliliter or 130 sporocysts per μg. It has been determined that such concentration allows for rapid analysis by a typical flow cytometer. Samples may be made more or less concentrated. Less concentrated samples require a longer time for a flow cytometer to analyze a sufficient minimum number of sporocysts. More concentrated samples may decrease accuracy depending on the concentration of such sample and the capacity of the particular flow cytometer used in connection with the instant invention. One skilled in the art will recognize the capacity of an instrument and can determine a sufficient concentration without undue experimentation.

When 80,000 sporocysts are present in a suspension totaling 0.6 ml in total volume and the sample is believed to contain a single species, it is preferred to pass the analyte sample through a flow cytometer until about 5,000 sporocysts have been analyzed. Thus, a putative single species sample or analyte is analyzed until 5,000 sporocysts have been counted. If a sample is believed to contain multiple species of sporocysts, such as an environmental sample or when testing the purity of a vaccine composition, it is preferred to analyze such sample until about 2,500 sporocysts are counted. Thus, a putative multiple species sample or analyte is analyzed until 2,500 sporocysts have been counted.

The present application has wide application. For example, the method can be used in the development of custom vaccination and/or treatment programs. In the development of treatment programs, samples can be obtained from infected animals individually or from representative animals within a group. Alternatively or in addition, samples can be obtained from the environment in which the animals are housed such as from bedding or litter, feed, water or equipment used. Samples can then be analyzed by the method described herein to determine the species of coccidial protozoa present, and if more than one species is present, the relative number of each species. Using this information, treatment programs can be designed using drugs which are effective against the species present. Methods for treatment of coccidiosis are well known in the art and can be found in standard veterinary references, for example, The Merck Veterinary Manual, 8th ed., Merck & Co., 1998.

In another embodiment, the present method can be used to design custom vaccination programs. Currently, vaccines against coccidiosis use live protozoa to confer immunity. Because, vaccination results in a sub-clinical case of coccidiosis, production following vaccination is adversely effected. It is anticipated that such adverse production effects may be reduced by limiting vaccination to those coccidial species actually present in the environment in which the animals are housed.

In this embodiment, a sample is taken from the environment in which the animals are housed or more preferably are to be housed. The sample can be obtained from any suitable source, including, but not limited to, bedding, floor litter, water, feed, equipment, cages, and feeders. Samples are then analyzed by the present method to determine which, if any, species of coccidial protozoa are present. Based on the results obtained, animals can then be vaccinated for those species present in the environment in which they are housed. Thus, the present method can be used to determine if coccidial protozoa are present in the environment at all. In those instances where it is found that the environment does not contain species of protozoa that are pathogenic to the species to be housed, the producer can opt to forego the expense and decreased productivity associated with vaccination.

The present method will also find use in quality control of coccidial vaccines. As discussed previously, many coccidial vaccines contain a mixture of several species of coccidial protozoa. These vaccines are produced by mixing coccidial protozoa isolated from host animals that have been infected with a pure line containing a single species. In order to guard against contamination with additional species, the coccidial protozoa obtained must be constantly monitored. Until the present invention, such monitoring was done by microscopic examination. The present invention provides a rapid and accurate method to assess the purity of coccidial protozoa produced from inoculated hosts. In addition, the method can be used to assess the species composition of the finished vaccine to monitor for mixing or other errors which may result in the species composition of the finished vaccine being different from what was intended.

### EXAMPLES

The following examples are intended to provide illustrations of the application of the present invention. The following examples are not intended to completely define or otherwise limit the scope of the invention.

### Example 1

### Identification Eimeria Species in Single Species Samples

E. tenella, E. acervulina and E. maxima oocysts were isolated and sporulated by methods described herein. The concentration of sporulated oocysts was determined using a hemacytometer and adjusted to approximately 1×10⁶ oocysts/ml for E. maxima and 2×10⁶ oocysts/ml for E. acervulina and E. tenella. Approximately 125 μl of 0.5 mm glass beads were added to microfuge tubes containing 1 ml of sporulated oocysts in PBS. Tubes were then vortexed on a FISHER Vortex (Fisher Scientific) at a speed setting of 7-8 for 5 minutes. After vortexing, a 10 μl sample was removed and checked for oocyst disruption. If virtually all of the oocysts were not disrupted, the sample was repeatedly vortexed at 1 minute intervals until virtually all the oocysts had been disrupted.

Following vortexing, the glass beads were allowed to settle and the supernatant placed in a clean microfuge tube. The beads were then washed by vortexing with 500 μl of PBS and the supernatant (wash) fluid collected and added to the previous supernatant. The tubes containing the combined supernatants were then centrifuged at approximately 9020×g for 2 minutes for tubes containing E. maxima or E. tenella, or 3 minutes for tubes containing E. acervulina. The resulting pellets were resuspended in 1 ml of 50% Percoll, centrifuged as above and the supernatant discarded. This process was repeated and the pellet resuspended in 200 μl of PBS and the concentration of sporocysts determined. For analysis by flow cytometry, approximately 80,000 sporocysts were place in each polystyrene sample tube and PBS added to bring the volume to approximately 600 μl.

Species identification was carried out using a Becton Dickinson FACScan flow cytometer with CellQuest software at a wavelength of 488 nm. COULTER Check Fluorosphere beads (2 drops in approximately 2 ml of PBS) were used to standardize the instrument so that the beads appeared in a specified region (R2) of a FCS vs SSC histogram (FIG. 1). Samples containing single species of sporocysts were then analyzed and data output regions defined. Region 1 (R1) was the area designation for E. acervulina sporocysts (FIG. 1), region 3 (R3) was the area designated for E. maxima sporocysts (FIG. 2), and region 4 (R4) was the area designated for E. tenella sporocysts (FIG. 3). After the data output regions were defined, the cytometer settings and the perimeters for each species were stored in data files and used for subsequent assays.

After the regions had been designated for each species, sporocysts were prepared for E. tenella, E. maxima and E. acervulina and analyzed as described above. For the individual species, the percent of the pattern of measurements for each species falling within the reference data output region associated with that species was 95.4%, 92.4% and 87.7% for E. acervulina, E. maxima, and E. tenella, respectively (Table 1). The total values in Table 1 vary from 100% due to background debris, the presence of intact oocysts not removed during sporocyst isolation, and the fact that there is some overlap between regions for E. acervulina and E. tenella.

TABLE 1

| Eimeria | % in E. acervulina | % in E. maxima | % in E. tenella |
|---|---|---|---|
| Species | Region (R1) | Region (R3) | Region (R4) |
| acervulina | 95.4 | 2.8 | 1.8 |
| maxima | 5.5 | 92.4 | 2.1 |
| tenella | 10.5 | 1.8 | 87.7 |

### Example 2

### Identification of Eimeria species in Mixed Samples

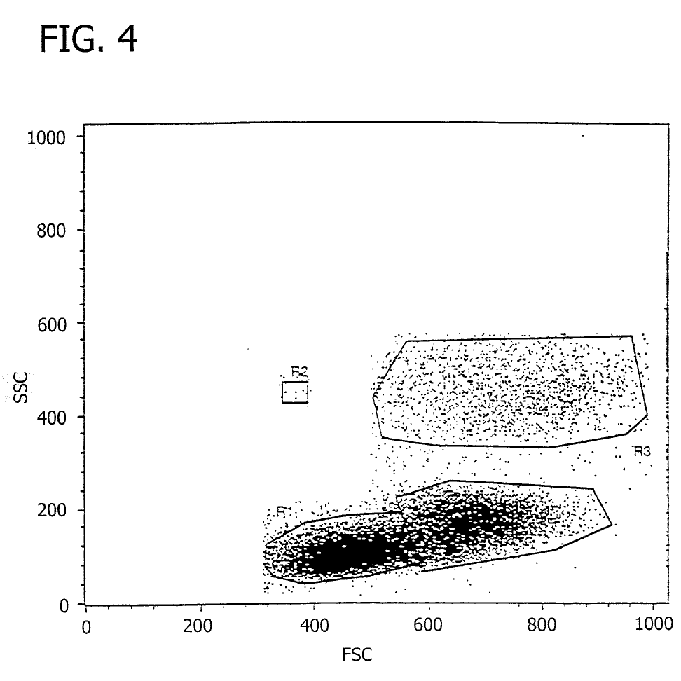

To examine whether mixed populations of Eimeria could be identified and quantitated, E. acervulina oocysts (2×10⁶), E. maxima oocysts (5×10⁵) and E. tenella oocysts (1×10⁶) were combined in PBS and sporocysts obtained as described in Example 1 for E. acervulina. The percent sporocysts for each species was determined by microscope examination (Table 2). The percent sporocysts for each species was then determined using the same sample by flow cytometry (FIG. 4). A second and third mixed species sample of oocysts were also prepared and analyzed by flow cytometry. Based on sporulation and recovery data, a theoretical percentage for each species of sporocyst was calculated. The results are given in Table 2 and show that percent of sporocysts for each species obtained by flow cytometry closely matched either the actual or theoretical values.

TABLE 2

| | | % Counts in | % Counts in | % Counts in |
|---|---|---|---|---|
| Sporocyst | | E. acervulina | E. maxima | E. tenella |
| Sample | Method | Region | Region | Region |
| First | Microscope | 53.1 | 11.7 | 36.7 |
| First | Cytometer | 55.7 | 18.3 | 26.0 |
| Second | Cytometer | 59.2 | 16.6 | 24.2 |
| Third | Cytometer | 51.8 | 10.2 | 38.1 |
| Average* | Cytometer | 55.6 | 15.0 | 29.4 |
| Theoretical | | 55.2 | 14.0 | 30.8 |

*Average of first, second, and third samples as determined by flow cytometry.

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for determining the number and species of coccidial protozoa in a sample comprising:
defining data output regions of a flow cytometer based on a measurement selected from the group consisting of forward light scatter, side light scatter, and fluorescence;
providing a sample of sporocysts from a population;
analyzing said sample using said flow cytometer; and
determining the number or percentage of each species of coccidial protozoa in said sample.

2. A method as set forth in claim 1, wherein said data output region is based on a measurement consisting of forward light scatter and side light scatter.

3. A method as set forth in claim 1, wherein said sample comprises from about 170 sporocysts to about 40 sporocysts per μg.

4. A method as set forth in claim 1, wherein said sample comprises from about 150 sporocysts to about 115 sporocysts per μg.

5. A method as set forth in claim 1, wherein said sample comprises about 130 sporocysts per μg.

6. A method as set forth in claim 1, wherein said data output regions are characterized as containing at least about 50% of the recorded sporocysts of the species of interest.

7. A method as set forth in claim 1, wherein said data output regions are characterized as containing at least about 85% of the recorded sporocysts of the species of interest.

8. A method as set forth in claim 1, wherein said data output regions are characterized as containing at least about 90% of the recorded sporocysts of the species of interest.

9. A method as set forth in claim 1, wherein said data output regions are characterized as containing at least about 95% of the recorded sporocysts of the species of interest.

10. A method as set forth in claim 1, wherein said protozoa are of the genus Eimeria.

11. A method as set forth in claim 10, wherein said protozoa are selected from the group consisting of E. tenella, E. acervulina and E. maxima.

12. A method as set forth in claim 1, further comprising staining said protozoa with at least one stain prior to analyzing said protozoa by flow cytometry.

13. A method as set forth in claim 12, wherein said stain is a fluorescent stain.

14. A method as set forth in claim 13, wherein said data output regions are further based on fluorescence intensity.

15. A method as set forth in claim 14, wherein said fluorescent stain is ethidium bromide.

16. A method for determining the species and number of protozoa of the genus Eimeria comprising:
defining data output regions of a flow cytometer based on forward light scatter and side light scatter, said data output regions characterized as containing at least about 85% of the recorded sporocysts of the species of interest;
providing a sample of sporocysts from a population;
analyzing said sample using said flow cytometer; and
determining the number of each species of coccidial protozoa in the sample.

17. A method for determining the species of coccidial protozoa in an environmental sample comprising:
defining data output regions of a flow cytometer based on forward light scatter and side light scatter, said data output regions characterized as containing at least about 50% of the recorded sporocysts of the species of interest;
isolating the protozoa from the sample;
if the protozoa are at the oocyst stage, inducing sporulation and isolating the sporocysts;
analyzing said sample using said flow cytometer; and
determining the number of each species of protozoa in the sample.

18. A method as set forth in claim 17, wherein said data output regions contain at least about 85% of the recorded sporocysts of the species of interest.

19. A method as set forth in claim 17, wherein said protozoa are of the genus Eimeria.

20. A method as set forth in claim 19, wherein said protozoa are selected from the group consisting of E. tenella, E. acervulina and E. maxima.

21. A method as set forth in claim 20, further comprising staining said protozoa with at least one stain prior to analyzing said protozoa by flow cytometry.

22. A method as set forth in claim 21, wherein said stain is a fluorescent stain.

23. A method as set forth in claim 22, wherein said data output regions are further based on fluorescence intensity.

24. A method as set forth in claim 22, wherein said fluorescent stain is ethidium bromide.

25. A method for determining the species of protozoa of the genus Eimeria in an environmental sample comprising:
defining data output regions of a flow cytometer based on forward light scatter and side light scatter, said data output regions characterized as containing at least about 85% of the recorded sporocysts of the species of interest;
isolating the protozoa from the sample;
if the protozoa are at the oocyst stage, inducing sporulation and isolating the sporocysts;
analyzing said sample using said flow cytometer; and
determining the number of each species of protozoa in the sample.

26. A method for treating an animal having a coccidial infection comprising:
obtaining a sample from the animal, a representative animal, or the environment in which the animal is housed;
isolating the coccidial protozoa from the sample;
if the protozoa are at the oocyst stage, inducing sporulation and isolating the sporocysts;
defining data output regions of a flow cytometer based on forward light scatter and side light scatter, said data output regions characterized as containing at least about 50% of the recorded sporocysts of the species of interest;
analyzing said sample using said flow cytometer; and
treating the animal for the species of coccidial protozoa identified.

27. A method as set forth in claim 26, wherein said data output regions contain at least about 85% of the recorded sporocysts of the species of interest.

28. A method as set forth in claim 26, wherein said protozoa are of the genus Eimeria.

29. A method as set forth in claim 26, wherein said protozoa are selected from the group consisting of E. tenella, E. acervulina and E. maxima.

30. A method as set forth in claim 26, further comprising staining said protozoa with at least one stain prior to analyzing said protozoa by flow cytometry.

31. A method as set forth in claim 30, wherein said stain is a fluorescent stain.

32. A method as set forth in claim 30, wherein said data output regions are further based on fluorescence intensity.

33. A method as set forth in claim 31 wherein said fluorescent stain is ethidium bromide.

34. A method for treating an animal infected with protozoa of the genus Eimeria comprising:
obtaining a sample from the animal, a representative animal, or the environment in which the animal is housed;
isolating the protozoa from the sample;
if the protozoa are at the oocyst stage, inducing sporulation and isolating the sporocysts; defining data output regions of a flow cytometer based on forward light scatter and side light scatter, said data output regions characterized as containing at least about 85% of the recorded sporocysts of the species of interest;
analyzing said sample using said flow cytometer; and
treating the animal for the species of protozoa identified.

35. A method for determining the number and species of coccidial protozoa in a pharmaceutical composition comprising:
obtaining a sample of the composition;
if the protozoa in the sample are at the oocyst stage, inducing sporulation and isolating the sporocysts;
defining data output regions of a flow cytometer based on forward light scatter and side light scatter, said data output regions characterized as containing at least about 50% of the recorded sporocysts of the species of interest;
analyzing said sample using said flow cytometer;
determining the number of each species of coccidial protozoa in the sample; and
calculating the number of each species in the pharmaceutical composition.

36. A method as set forth in claim 35, wherein said data output regions contain at least about 85% of the recorded sporocysts of the species of interest.

37. A method as set forth in claim 35, wherein said protozoa are of the genus Eimeria.

38. A method as set forth in claim 37, wherein said protozoa are selected from the group consisting of E. tenella, E. acervulina and E. maxima.

39. A method for determining the species of coccidial protozoa in a sample comprising:
passing an analyte comprising a suspension of unknown sporocysts of said protozoa sample in a liquid medium through a flow cytometer;
impinging light on said analyte passing through said flow cytometer;
measuring at least one characteristic of said sample, said characteristic being selected from the group consisting of forward flow light scatter, side light scatter, and fluorescence;
determining a pattern of measurements obtained from said analyte with respect to said at least one characteristic; and
comparing said pattern of measurements for said analyte with a reference pattern of measurements with respect to said at least one characteristic as derived from measurements taken in passing at least one reference suspension through a flow cytometer;
and determining the species of protozoa in said analyte.

40. A method as set forth in claim 39 wherein said at least one reference suspension comprises substantially the same medium as said analyte, and said reference pattern is obtained by passing said at least one reference suspension through said flow cytometer and impinging light thereon under reference conditions comprising concentration, sheath fluid, flow rate, temperature, wavelength of the laser, and power setting of the laser that are substantially the same as the conditions under which said analyte is passed through said flow cytometer.

41. A method as set forth in claim 40 wherein said at least one characteristic of said reference pattern is the same as a characteristic with respect to which a pattern of measurements is determined for said analyte.

42. A method as set forth in claim 41 wherein said reference pattern comprises a composite pattern of measurements derived from a plurality of reference suspensions each containing the same sporocyst of known identity.

43. A method as set forth in claim 42 wherein said plurality of reference suspensions comprises about 10 reference suspensions.

44. A method as set forth in claim 42 wherein said plurality of reference suspensions comprises about 20 reference suspensions.

45. A method as set forth in claim 42 wherein said plurality of reference suspensions comprises about 30 reference suspensions.

46. A method as set forth in claim 41 wherein the pattern of measurements determined for said analyte comprises the frequency distribution of measurements over a range of values for said at least one characteristic; and said determined frequency distribution is compared with a reference pattern comprising a known frequency distribution with respect to said at least one characteristic for at least one reference suspension over the same range of values as obtained when said at least one reference suspension is passed through said flow cytometer under said reference conditions.

47. A method as set forth in claim 46 wherein said pattern of measurements determined for said analyte comprises the frequency distribution of measurement values over a plurality of ranges of values for said at least one characteristic; and said determined frequency distribution is compared with a reference pattern comprising the known frequency distribution with respect to said at least one characteristic for said at least one reference suspension over the same plurality of ranges of values as obtained when said at least one reference suspension is passed through said flow cytometer under said reference conditions.

48. A method as set forth in claim 46 wherein said pattern of measurements determined for said analyte is compared with a plurality of reference patterns obtained from a plurality of reference suspensions containing different sporocysts of known identity, each of the reference patterns comprising the frequency distribution with respect to said characteristic over a range of values as obtained when at least one reference suspension containing a particular known sporocyst is passed through said flow cytometer under said reference conditions.

49. A method as set forth in claim 48 wherein the pattern of measurements determined for said analyte comprises a frequency distribution of combinations of measured values of a plurality of characteristics of said analyte over a combination of ranges of values for said plurality of characteristics; and said determined frequency distribution is compared with a plurality of reference patterns for a plurality of different known sporocysts, each of said reference patterns comprising a frequency distribution of combinations of measured values of the same plurality of characteristics that are measured in obtaining the frequency distribution for said analyte.

50. A method as set forth in claim 49 wherein each of said reference patterns comprises a composite pattern of measurements derived from a plurality of patterns obtained from a plurality of reference suspensions each containing a particular sporocyst of known identity.

51. A method as set forth in claim 49 wherein each of said patterns of measurements determined for said analyte and said reference patterns constitutes a data output region of said flow cytometer within a field having the dimensions of the values of said plurality of measured characteristics, and wherein when at least 85% of the combinations of data values as measured by the flow cytometer for said plurality of characteristics of said analyte are contained within one of said reference pattern data output regions, an indication is provided that said analyte comprises sporocysts of an identity correlating to the known identity of the sporocysts contained in the at least one reference suspension from which said reference data output region is generated.

52. A method as set forth in claim 51 wherein the plurality of reference data output regions comprises a region which encompasses at least about 85% of the of the combinations of measured values of characteristics of said analyte.

53. A method as set forth in claim 52 wherein the plurality of reference data output regions comprises a region which encompasses at least about 90% of the of the combinations of measured values of characteristics of said analyte.

54. A method as set forth in claim 53 wherein the plurality of reference data output regions comprises a region which encompasses at least about 95% of the of the combinations of measured values of characteristics of said analyte.

55. A method as set forth in claim 51 wherein said analyte contains a plurality of unknown species of sporocysts.

56. A method as set forth in claim 51 wherein said analyte contains a putative plurality of species of sporocysts and said method is utilized to assess purity of said analyte.

57. A method as set forth in claim 39 wherein said pattern of determined measurements of said analyte comprises a combination of measurements of a plurality of different characteristics of said analyte as measured in said flow cytometer; and said reference pattern comprises a combination of measurements of the same plurality of different characteristics as obtained when at least one reference suspension is passed through said flow cytometer.

58. A method as set forth in claim 57 wherein said determined pattern is compared with a plurality of reference patterns obtained from a plurality of reference patterns obtained for different sporocysts of known identity, each of said reference patterns comprising combinations of measured values of the same plurality of different characteristics.

59. A method as set forth in claim 58 wherein said sample contains a plurality of unknown sporocysts, and said determined pattern is compared with a plurality of reference patterns obtained from a plurality of reference suspensions containing different sporocysts of known identity, each of said reference patterns comprising combinations of measured values of the same plurality of different characteristics, whereby said comparison provides information on both the identity and the concentration of the sporocysts in said analyte.

60. A method as set forth in claim 59 further comprising analyzing a putative multiple species analyte until 2,500 recorded events are attained.

61. A method as set forth in claim 59 further comprising analyzing a putative single species analyte until 5,000 recorded events are attained.

62. A method as set forth in claim 59 further comprising sorting at least one population of sporocysts from said analyte, said sorted at least one population being characterized as having a said determined pattern of measurements wherein at least about 85% of said pattern of measurements is encompassed by a reference pattern of measurements.

63. A method for determining the species of coccidial protozoa in a sample comprising:
passing an analyte comprising a suspension of unknown sporocysts of said protozoa sample in a liquid medium through a flow cytometer;
impinging light on said analyte passing through said flow cytometer;
measuring at least one characteristic of said sample, said characteristic being selected from the group consisting of forward flow light scatter, side light scatter, and fluorescence;
determining a pattern of measurements obtained from said analyte with respect to said at least one characteristic; and
comparing said pattern of measurements for said analyte with a reference pattern of measurements with respect to said at least one characteristic as derived from measurements taken in passing known sporocysts through a flow cytometer;
and determining the species of protozoa in said analyte.

64. A method as set forth in claim 63 wherein said reference pattern is obtained by passing said known sporocysts through said flow cytometer in substantially the same medium as said analyte, and impinging light on said known sporocysts under reference conditions comprising concentration, sheath fluid, flow rate, temperature, wavelength of the laser, and power setting of the laser that are substantially the same as the conditions under which said analyte is passed through said flow cytometer.

65. A method as set forth in claim 64 wherein said at least one characteristic of said reference pattern is the same as a characteristic with respect to which a pattern of measurements is determined for said analyte.

66. A method as set forth in claim 65 wherein said reference pattern comprises a composite pattern of measurements derived from a plurality of reference suspensions each containing the same sporocyst of known identity.

67. A method as set forth in claim 65 wherein the pattern of measurements determined for said analyte comprises the frequency distribution of measurements over a range of values for said at least one characteristic; and said determined frequency distribution is compared with a reference pattern comprising a known frequency distribution with respect to said at least one characteristic for known sporocysts over the same range of values as obtained when said known sporocysts are passed through said flow cytometer under said reference conditions.

68. A method as set forth in claim 67 wherein said pattern of measurements determined for said analyte comprises the frequency distribution of measurement values over a plurality of ranges of values for said at least one characteristic; and said determined frequency distribution is compared with a reference pattern comprising the known frequency distribution with respect to said at least one characteristic for said known sporocysts over the same plurality of ranges of values as obtained when said known sporocysts are passed through said flow cytometer under said reference conditions.

69. A method as set forth in claim 67 wherein said pattern of measurements determined for said analyte is compared with a plurality of reference patterns obtained from passing a plurality of known sporocysts through said flow cytometer, each of the reference patterns comprising the frequency distribution with respect to said characteristic over a range of values as obtained when said known sporocysts are passed through said flow cytometer under said reference conditions.

70. A method as set forth in claim 69 wherein the pattern of measurements determined for said analyte comprises a frequency distribution of combinations of measured values of a plurality of characteristics of said analyte over a combination of ranges of values for said plurality of characteristics; and said determined frequency distribution is compared with a plurality of reference patterns for a plurality of different known sporocysts, each of said reference patterns comprising a frequency distribution of combinations of measured values of the same plurality of characteristics that are measured in obtaining the frequency distribution for said analyte.

71. A method as set forth in claim 70 wherein each of said reference patterns comprises a composite pattern of measurements derived from a plurality of patterns obtained from a plurality of known sporocysts.

72. A method as set forth in claim 70 wherein each of said patterns of measurements determined for said analyte and said reference patterns constitutes a data output region of said flow cytometer within a field having the dimensions of the values of said plurality of measured characteristics, and wherein when at least 85% of the combinations of data values as measured by the flow cytometer for said plurality of characteristics of said analyte are contained within one of said reference pattern data output regions, an indication is provided that said analyte comprises sporocysts of an identity correlating to the identity of the known sporocysts from which said reference data output region is generated.

73. A method as set forth in claim 72 wherein the plurality of reference data output regions comprises a region which encompasses at least about 85% of the of the combinations of measured values of characteristics of said analyte.

74. A method as set forth in claim 73 wherein the plurality of reference data output regions comprises a region which encompasses at least about 90% of the of the combinations of measured values of characteristics of said analyte.

75. A method as set forth in claim 74 wherein the plurality of reference data output regions comprises a region which encompasses at least about 95% of the of the combinations of measured values of characteristics of said analyte.

76. A method as set forth in claim 72 wherein said analyte contains a plurality of unknown species of sporocysts.

77. A method as set forth in claim 72 wherein said analyte contains a putative plurality of species of sporocysts and said method is utilized to assess purity of said analyte.

78. A method as set forth in claim 63 wherein said pattern of determined measurements of said analyte comprises a combination of measurements of a plurality of different characteristics of said analyte as measured in said flow cytometer; and said reference pattern comprises a combination of measurements of the same plurality of different characteristics as obtained when known sporocysts are passed through said flow cytometer.

79. A method as set forth in claim 78 wherein said determined pattern is compared with a plurality of reference patterns obtained from a plurality of reference patterns obtained for different sporocysts of known identity, each of said reference patterns comprising combinations of measured values of the same plurality of different characteristics.

80. A method as set forth in claim 79 wherein said sample contains a plurality of unknown sporocysts, and said determined pattern is compared with a plurality of reference patterns obtained from a plurality of reference suspensions containing different known sporocysts, each of said reference patterns comprising combinations of measured values of the same plurality of different characteristics, whereby said comparison provides information on both the identity and the concentration of the sporocysts in said analyte.

81. A method as set forth in claim 80 further comprising analyzing a putative multiple species analyte until 2,500 recorded events are attained.

82. A method as set forth in claim 80 further comprising analyzing a putative single species analyte until 5,000 recorded events are attained.

83. A method as set forth in claim 80 further comprising sorting at least one population of sporocysts from said analyte, said sorted at least one population being characterized as having a said determined pattern of measurements wherein at least about 85% of said pattern of measurements is encompassed by a reference pattern of measurements.
